(19) United States
(12) Patent Application Publication
(10) Pub. No.: US 2005/0196315 A1
(43) Pub. Date: Sep. 8, 2005

(54) Modular sterilization system

(75) Inventors: Sergei Babko-Malyi, West Windsor, NJ (US); Richard Crowe, Hazlet, NJ (US)

Correspondence Address: DARBY & DARBY P.C., P. O. BOX 5257, NEW YORK, NY, 10150-5257, US

(73) Assignee: Plasmasol Corporation, Hoboken, NJ (US)

(21) Appl. No.: 11/042,359

(22) Filed: Jan. 24, 2005

Related U.S. Application Data
(60) Provisional application No. 60/538,742, filed on Jan. 22, 2004.

(51) Int. Cl. A61L 2/02

(52) U.S. Cl. 422/23; 422/292; 422/186.05

(57) ABSTRACT

A modular sterilization system including a modular sterilization section divided into a plurality of compartments. The system further includes a plurality of units, each unit being closable and received within one of the compartments of the modular sterilization section. A gas discharge generator is disposed in fluid communication with each unit to generate a weakly ionized gas that sterilizes the object(s) to be treated that are housed therein. Power is provided independently to only those compartments in which a corresponding unit has been properly installed. An electric field is thereby generated only in the generators of those units that have received power. As a result, the weakly ionized gas is emitted from the generator in which an electric field has been created in situ of an object to be treated.

### CROSS-REFERENCE TO RELATED APPLICATIONS

This application claims the benefit of U.S. Provisional Application No. 60/538,742, filed Jan. 22, 2004, which is hereby incorporated by reference.

### BACKGROUND OF THE INVENTION

1. Field of the Invention

The present invention relates to sterilization of an object and, in particular, to a modular system for sterilizing, disinfecting or decontamination of objects (e.g., medical instruments) utilizing non thermal plasma and associated chemical methods.

2. Description of Related Art

For health and safety reasons it is necessary in various applications, the most common being medical and food applications, to reduce the number of microorganisms on an object or item. The terms sterilization, disinfection and decontamination are just some of the words often used to classify or categorize a particular chemical substance or process based on its ability to reduce the level of microorganisms living on an item. The sterility assurance level (SAL) is the expected probability of an item being non-sterile (i.e., capable of sustaining microorganisms on the item) after exposure to a sterilization process. A preferred SAL for medical devices is 10⁻³ (one in a thousand) for less critical devices and 10⁻⁶ (one in a million) for more critical and invasive devices such as an endoscope. Despite the fact that certain standards set appropriate ranges to specifically define and distinguish between the terms sterilization, disinfectant, and decontamination often these terms are used interchangeably by those of skill in the art. Moreover, with changing technology, the definitions and, in particular, the SAL for each may vary over time for a particular industry or application. Accordingly, the term “sterilization” used herein is broadly defined as a process that reduces the likelihood of microorganisms living on an item exposed to the process. By varying the sterilization process the SAL may be increased or decreased, as necessary, for the particular application.

The sterilization process can occur via a physical and/or a chemical process. Batch processing using a variety of techniques to achieve sterilization is the prevalent method used today. Two of the most common techniques include the use of steam autoclaving and chemical sterilization. Due to the nature of the systems and hazards associated with some of the sterilization chemicals, it is common to process instruments on a batch cycle basis, placing containers into large sterilization chambers for processing. Accordingly, the housing of the sterilizer has heretofore been designed to accommodate any number of one or more trays. Despite the inherent advantage of improved efficiency associated with designing the sterilization chamber to accommodate multiple trays at a single time, this feature has numerous drawbacks. Such larger loads require longer sterilization times and larger volumes of liquid. This is inefficient when less than the full capacity of trays is loaded into the sterilization chamber. Another disadvantage associated with placing a plurality of trays or containers into a conventional sterilizer housing concerns their placement or stacking. In order to ensure proper sterilization of the objects, the trays or containers must be properly stacked in accordance with predetermined guidelines provided by each manufacturer. It would be advantageous to design an improved user friendly sterilization system with failsafe loading that inherently would satisfy the manufacturer's guidelines. It is therefore desirable to develop an improved modular sterilization system able to accommodate existing sterilization trays that overcome the disadvantages associated with conventional sterilization systems.

Traditional chemical sterilants (such as ethylene oxide) are typically injected into a sterilization chamber. After sterilization is completed, the chamber is evacuated and the chemical sterilant is exhausted to a recovery system either for reprocessing or disposal. Due to the hazardous nature of many of these chemicals, such as ethylene oxide which has been classified by national health and safety organizations to be carcinogenic and neurotoxic, special handling procedures are required for both pre-sterilization as well as post-sterilization. Furthermore, safety concerns require the constant monitoring of the sterilization facility for leaks of the chemical sterilant. In addition, some chemical sterilants (such as ethylene oxide) are highly combustible and thus often are diluted with carbon dioxide or freon which destroy the ozone layer.

Conventional sterilizers (steam autoclave, Ethylene Oxide, Sterrad™) are typically operated by placing a plurality of trays within the sterilization chamber. These sterilization systems require the trays to be isolated from their surrounding environment via a permeable filter media. Generally, the trays are wrapped in the permeable media. This isolation technique is necessary to maintain the sterility of the items when removing the tray from the sterilizer chamber for use elsewhere. The permeable filter media allows the sterilizing agent (e.g., chemical or steam) to diffuse and contact the items in the tray to be sterilized while substantially blocking the transfer of particles outside the media from reaching the contents protected therein.

It is therefore desirable to develop an improved sterilization system able to develop an in situ transient biocide within the sterilization chamber that: (i) has a relatively short lifespan outside the chamber, (ii) employs non toxic and safe to handle precursors, and (iii) eliminates the need for use of a permeable filter media.

### SUMMARY OF THE INVENTION

The present invention is an improved sterilization system that is more efficient while reducing health and environmental hazards by employing biologically active yet relatively short living sterilant species produced as a byproduct during the generation of non-thermal plasma. Furthermore, the present inventive process and system improves overall sterilization efficiency by employing a modular design that reduces wasted power and additives.

Specifically, the present invention is directed to a method of sterilization of objects such as but not limited to medical instruments. Active sterilizing species are generated as a result of passing organic compounds through a weakly ionized gas (most typical is a non-thermal plasma). Due to the relatively short lifetime of the active sterilizing species their sterilization capabilities are greatest or most effective while in the vicinity of the gas discharge device. At the same time, due to its relatively short lifetime the active sterilization species decompose rapidly into benign byproducts. This decomposition characteristic is useful in general where sterilization must be realized with minimal health and environmental hazards. The gas discharge generator is in fluid communication with the unit housing the object to be treated. The byproducts of the plasma-chemical reactions (such as ozone, nitrogen oxides, organic acids, aldehydes) that are commonly present in the discharge afterglows in trace amounts may be captured in an off-gas treatment system based on adsorption, catalysis or other processes typically used for removal of these byproducts from air.

To improve the sterilization efficiency rate, an organic based reagent may be injected through the electrodes and/or directly into a discharge region. This organic based reagent serves as both precursor to increase production of active sterilizing species while transporting the active sterilizing species with the fluid flow to the desired contaminated regions or surfaces to be treated. The resulting chemical reaction is able to be generated and directed in situ to particular regions or areas of an object to be sterilized or decontaminated without requiring a negative pressure in the chamber using a vacuum pump. As soon as power to the discharge device is turned off, the active sterilizing species ceases to be generated and the objects may be immediately removed from the chamber without further delay.

Rather than placing a plurality of containers, assemblies and materials into a relatively large sterilization chamber the present invention exposes the objects to be treated to a weakly ionized gas within individual units received in compartments. This allows for flexible expansion of capacity to meet the specific needs of the sterilization environment whether treating a single object or hundreds of objects.

To achieve these goals the present invention is directed to a modular sterilization system including a modular sterilization section divided into a plurality of compartments. The system further includes a plurality of units, each unit being closable and dimensioned in size and shape to complement and be received within one of the compartments of the modular sterilization section. A gas discharge generator disposed in fluid communication with each unit generates a weakly ionized gas that sterilizes the object(s) to be treated that are housed therein. Power is provided independently to only those compartments in which a corresponding unit has been properly installed. An electric field is thereby generated only in the gas discharge generators of those units that have received power. As a result, the weakly ionized gas is emitted from the generator in which an electric field has been created and in situ of an object to be treated.

### BRIEF DESCRIPTION OF THE DRAWINGS

The foregoing and other features of the present invention will be more readily apparent from the following detailed description and drawings of illustrative embodiments of the invention wherein like reference numbers refer to similar elements throughout the several views and in which:

FIG. 1 is a perspective view of an exemplary six unit modular sterilization section in accordance with the present invention;

FIG. 2 is a perspective view of an exemplary modular sterilization system including two modular subsections connected together in accordance with the present invention;

FIG. 3A is a perspective view of a single exemplary tray and lid in accordance with the present invention with the lid removed from the tray showing the interior surface of the lid; and

FIG. 3B is a perspective view of the single tray and lid of FIG. 3A with the lid closed on the tray.

### DETAILED DESCRIPTION OF THE PREFERRED EMBODIMENTS

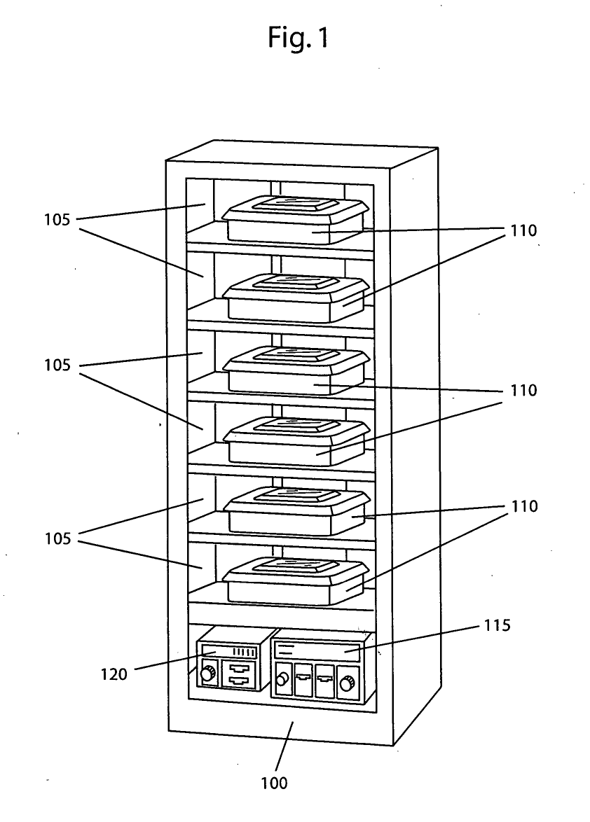

By way of example, the present invention shows and describes a modular sterilization system for use in a medical sterilization application. It is, however, contemplated and within the intended scope of the present invention to employ the modular sterilization system in other applications employing sterilization techniques such as, but not limited to, the handling of food. An exemplary six unit modular sterilization section 100 in accordance with the present invention is shown in FIG. 1. The sterilization section may be designed, as desired, to accommodate any number of one or more units. In the present invention, the term “units” is generically used to describe any closable container such a tray with a lid, a closable box or a closable bag. Each unit may be adapted in size and shape based on the size and shape of the particular objects being treated. The modular sterilization section is designed with one or more compartments 105 adapted in size and shape to preferably receive only one unit 110. Thus, the capacity of the modular sterilization section 100 is limited by the number of compartments 105. By way of example, the modular sterilization section shown in FIG. 1 has six compartments 105 capable of accommodating six or less units 110, one compartment being adapted to receive a single unit. A control module 115 is installed to provide electricity (either DC or AC) to and vary the parameters for each of the individual units 110. For instance, control module 115 may independently control for each unit 110 the type and quantity of an organic based reagent introduced therein, the period for sterilization, the sterilization cycles, and/or power level. It may also be desirable, but not necessary, to have the control module 115 monitor one or more parameters or conditions such as time of operation or unit status. Each unit, in turn, may be further divided or subdivided into nested compartments or sub compartments the sterilization parameters or conditions for each which again may be independently and individually controlled by the control module 115.

In a preferred embodiment, each unit 110 is adapted to produce a weakly ionized gas, e.g. plasma therein. A weakly ionized gas is a partially ionized gas composed of ions, electrons, and neutral species. This state of matter is produced by relatively high temperatures and/or relatively strong electric fields either constant (DC) or time varying (e.g., AC) electromagnetic fields. The weakly ionized gas is produced when free electrons are energized by electric fields in a background of neutral atoms/molecules. These electrons cause electron atom/molecule collisions which transfer energy to the atoms/molecules and form a variety of species which may include photons, metastables, atomic excited states, free radicals, molecular fragments, electrons, and ions. The neutral gas becomes partially or fully ionized and is able to conduct electric currents. The species are chemically active and/or able to physically modify the surface of materials and may therefore serve to form new chemical compounds and/or modify existing compounds.

The use of weakly ionized gas such as plasma as a means for sterilization is well known. Any type of conventional gas discharge reactor configuration may be used to generate the weakly ionized gas such as a corona or barrier discharge plasma reactor. Several inventive generator configurations assigned to the same company as that of the present invention are disclosed in issued and pending related patent applications and are well suited for use in the present invention. Specifically, a capillary discharge plasma generator configuration is shown in U.S. Pat. No. 6,818,193, issued on Nov. 16, 2004, entitled “Segmented Electrode Capillary Discharge Non-Thermal Plasma Apparatus and Process for Promoting Chemical Reactions”. Alternative gas discharge configurations disclosed in pending applications include a Slot Discharge (described in U.S. Ser. No. 10/371,243, filed on Feb. 19, 2003, which claims the benefit of U.S. Provisional Application No. 60/358,340, filed Feb. 19, 2002) and Capillary-in-Ring Electrode Non-Thermal Plasma Generator and Method for Using the Same (described in provisional U.S. application Ser. No. 60/538,743, filed on Jan. 22, 2004, the non-provisional application of which was filed on Jan. 24, 2005 and assigned U.S. Ser. No. ______ (Attorney Docket No. 02790/100M780-US1) configurations. Each of these pending and issued patents are herein incorporated by reference in their entirety. These plasma generator configurations substantially suppress discharge transitions to the arc mode while increasing the surface area of the discharge or emissions from the reactor, however, the present invention may be modified for application using any type of gas discharge generator.

The generation of the weakly ionized gas requires the application of an electric field to an electrode. Thus, a modular sterilization section 100 adapted to sterilize objects in situ by exposure to a gas discharge requires that each compartment 105 be electrically connected to receive energy from a power source 120 in order to generate the electric field. Correspondingly, each unit also contains electronic circuitry connected to the electrode. In a preferred embodiment, an interface or adapter, for example, complementary male and female plugs, are provided on the respective unit 110 and corresponding compartment 105 so that when the unit is inserted into a compartment the male and female connectors automatically align to complete the connection. Alternatively, cable may extend from the compartment to be manually connected to a complementary port or outlet of the unit.

The electric field will only be applied to those compartments for which the circuit has been closed or completed. That is, only those compartments loaded with and properly connected to an associated unit will generate an electric field. All empty compartments (i.e., those for which no unit has been inserted or the circuit has not been closed or completed) will not draw energy from the power source because the electrical circuit remains open. In this regard, the efficiency of the modular sterilization system is improved over that of the prior art in that only the necessary amount of power need to sterilize the particular number of loaded trays will be required.

To increase concentrations of generated chemically active species, e.g., ions and free radicals, thereby accelerating and improving the overall destruction rates of undesirable chemical and/or biological contaminants an organic based reagent may be introduced into the plasma or weakly ionized gas, as described in detail in the pending application entitled “System and Method for Injection of an Organic Based Reagent in Weakly Ionized Gas to Generate Chemically Active Species”, U.S. patent application Ser. No. 10/407,141, filed on Apr. 2, 2003 (which claims the benefit of U.S. Provisional Application No. 60/369,654, filed Apr. 2, 2002) (having the same assignee as the present invention), said application being incorporated by reference in its entirety. The organic based reagent may be a combination of an organic additive (e.g., an alcohol or ethylene) mixed with an oxidizer (e.g., oxygen) prior to being introduced in the weakly ionized gas. Alternatively, the organic based reagent may be the injection of an organic additive alone in the weakly ionized gas while in the presence of air (non vacuum chamber) that inherently contains oxygen and serves as the oxidizer. Also, the organic based reagent may comprise an organic additive that itself includes an oxidizing component such as ethanol. In this situation the oxidizing component of the organic component when injected into the weakly ionized gas forms hydroxyl radicals, atomic oxygen or other oxidizing species that may be sufficient to eliminate the need for a supplemental oxidizer. Regardless of the organic based reagent used, the organic additive reacts with the oxidizer while in the presence of weakly ionized gas to initiate the production of chemically active species. The modular sterilizer may be adapted to be connected to a supply source for receiving the organic based reagent independently into each of the units 110. This supply source may be disposed within or outside of the housing of the modular sterilization section depending on its size.

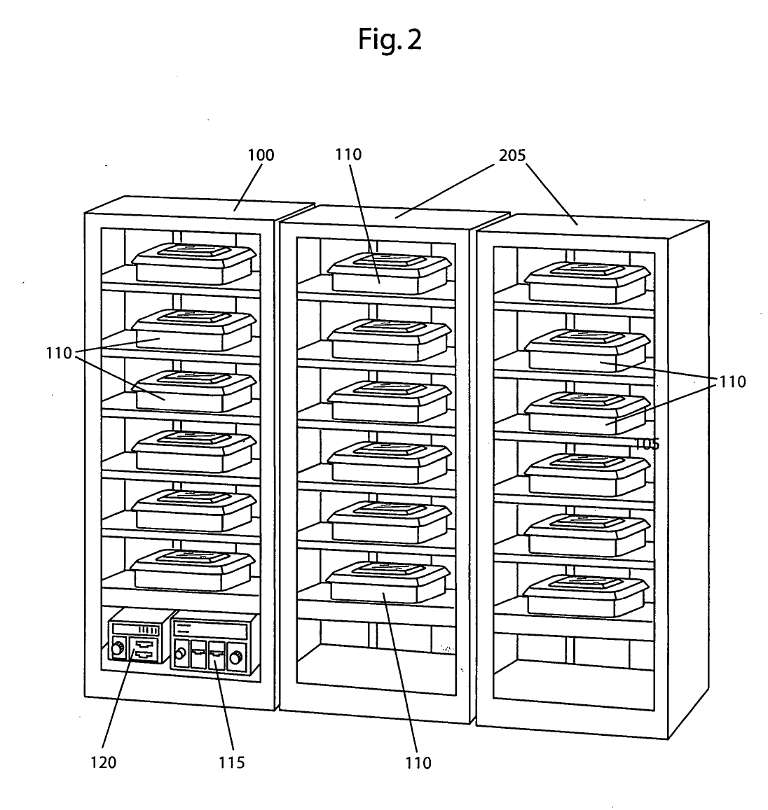

As shown in FIG. 2, two or more slave modular sterilization sections 205 may be connected to the master modular sterilization section 100 to increase its capacity and together form a modular sterilization grid. In the example shown in FIG. 2, three modular sterilization sections (two slave units 205 and one master unit 100) are connected together to form a modular sterilization system or grid. The modular sterilization sections may be connected on any one or more of its sides to another modular sterilization section. Each of the multiple modular sterilization sections may have the same capacity, as shown in FIG. 2 wherein each modular sterilization section has a six unit capacity. Alternatively, different capacity modular sterilizations sections may be connected together to form a modular sterilization system or grid.

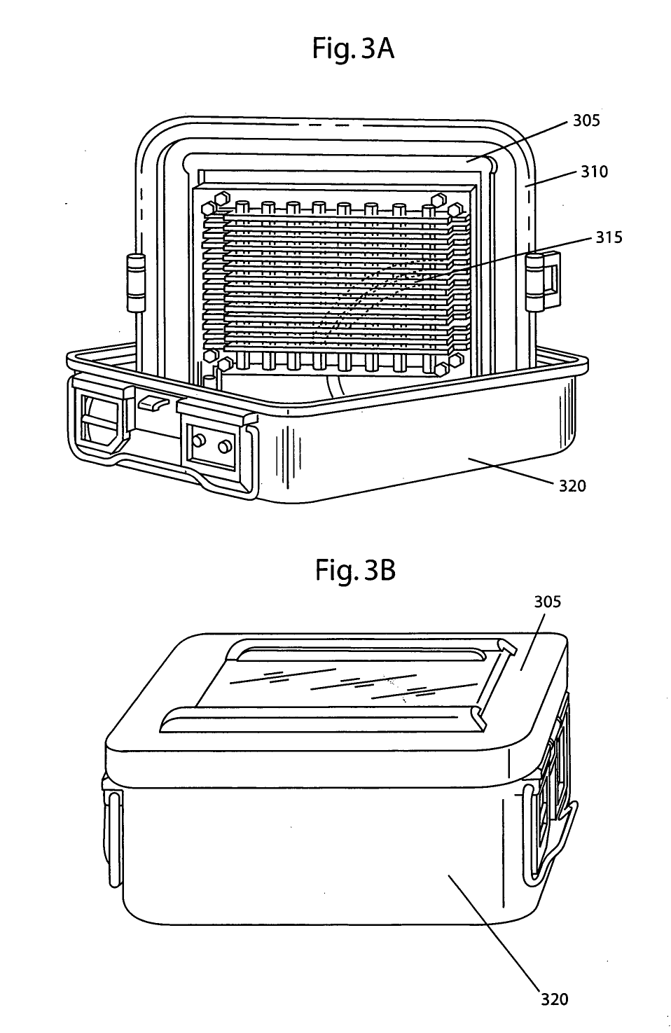

FIGS. 3A & 3B depict and exemplary unit 110 configured as an assembled tray and complementary lid. Lid 305 can be fabricated from a variety of materials (metallic, non-metallic, etc) and is form fit to a mating tray 320. A negative fit device (typically a gasket) 310 is preferably employed to form a seal, keeping the transient biocide within the unit 110 to ensure sterility of the contents therein after the process is complete and the unit removed from the system or grid 100. A gas discharge generator 315 for producing a weakly ionized gas is disposed to generate the transient biocide in the interior of the unit. The generator 315 shown in FIGS. 3A & 3B is a capillary configuration or structure as described in U.S. Pat. No. 6,818,193, however, any type of gas discharge generator 315 may be used. Furthermore, the generator shown in FIGS. 3A & 3B have incorporated the gas discharge generator in the top or lid of the unit. Positioning of the gas discharge generator may be modified so long as the weakly ionized gas is emitted into the interior of the unit with the object to be treated directly exposed to the discharge or emission.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

All references, publications, pending and issued patents are herein each incorporated by reference in their entirety.

1. A modular sterilization system, comprising:
at least one modular sterilization section divided into a plurality of compartments;
a plurality of units, each unit dimensioned in size and shape to complement and be received within one of the plural compartments of the modular sterilization section; and
a gas discharge generator associated with and disposed in fluid communication with each unit for generating a weakly ionized gas into an interior of the unit.

2. The system in accordance with claim 1, wherein each unit is a closable container.

3. The system in accordance with claim 2, wherein the unit includes a tray and a mating lid.

4. The system in accordance with claim 3, wherein the gas discharge generator is incorporated in the lid of the unit.

5. The system in accordance with claim 2, wherein the unit is a closable bag.

6. The system in accordance with claim 1, wherein each compartment is adapted to be independently connectable to a power source.

7. The system in accordance with claim 6, wherein the unit and the compartment have adapters complementary in shape to engage one another and draw energy from the power source when the unit is properly installed in the compartment.

8. The system in accordance with claim 6, wherein the power source provides power only to those compartments in which an associated unit has been properly installed.

9. The system in accordance with claim 1, further comprising a control module for independently setting conditions for each of the compartments.

10. The system in accordance with claim 1, wherein the system includes at least two modular sterilization sections including a master sterilization section and a slave sterilization section, the modular sterilization sections being connectable to one another to form a grid.

11. A method for sterilization using a modular sterilization system including at least one modular sterilization section divided into a plurality of compartments; a plurality of units, each unit dimensioned in size and shape to complement and be received within one of the plural compartments of the modular sterilization section; and a gas discharge generator associated with and disposed in fluid communication with each unit for generating a weakly ionized gas into an interior of the unit, wherein the method comprises the steps of:
providing power independently to only those compartments in which a corresponding unit has been properly installed;
generating an electric field only in the generators of those units that have received power; and
emitting in situ of an object to be treated the weakly ionized gas from the generator in which an electric field has been created.

12. The method in accordance with claim 11, further comprising the step of independently varying at least one condition of each of the units via a control module.

13. The method in accordance with claim 12, wherein the at least one condition is at least one of: (i) type and quantity of an organic based reagent introduced therein; (ii) period for sterilization; (iii) sterilization cycles; and (iv) power level.

14. The method in accordance with claim 11, wherein the unit is a closable container.

15. The method in accordance with claim 14, wherein the unit includes a tray and a mating lid.

16. The method in accordance with claim 15, wherein the gas discharge generator is incorporated in the lid of the unit.

17. The system in accordance with claim 2, wherein the unit is a closable bag.

18. The method in accordance with claim 11, wherein the unit and the compartment have adapters complementary in shape to engage one another and draw energy from the power source when the unit is properly installed in the compartment.

19. The method in accordance with claim 11, wherein the system includes at least two modular sterilization sections including a master sterilization section and a slave sterilization section, the modular sterilization sections being connectable to one another to form a grid.

20. The method in accordance with claim 11, wherein the unit is subdivided.
